(19) United States
(12) Patent Application Publication
(10) Pub. No.: US 2023/0170095 A1
(43) Pub. Date: Jun. 1, 2023

(54) GAIT-BASED MOBILITY ANALYSIS

(71) Applicant: INTERNATIONAL BUSINESS MACHINES CORPORATION, ARMONK, NY (US)

(72) Inventors: TIAN HAO, White Plains, NY (US); JEFFREY L. ROGERS, Briarcliff Manor, NY (US); Pritish Ranjan Parida, Cortlandt Manor, NY (US)

(21) Appl. No.: 17/456,902

(22) Filed: Nov. 30, 2021

(51) Int. Cl. G16H 50/30; A61B 5/024; A61B 5/11

(52) U.S. Cl. CPC G16H 50/30; A61B 5/024; A61B 5/112

(57) ABSTRACT

A method, a structure, and a computer system for assessing user mobility. The exemplary embodiments may include collecting heart rate data and acceleration data corresponding to a user while the user is not performing one or more validated fitness assessment tests and extracting one or more features from the heart rate data and the acceleration data. The exemplary in embodiments may further include calculating one or more validated fitness assessment scores of the user based on applying a model to the one or more features and projecting a mobility of the user based on the one or more validated fitness assessment scores.

### BACKGROUND

The exemplary embodiments relate generally to assessing user mobility, and more particularly to assessing user mobility via gait analysis.

Mobility refers to one's ability to move freely and easily. Physiologically, mobility is a manifestation of a functional integration of skeletal, muscular, nervous, circulatory, and respiratory systems. Thus, mobility may represent critical clinical evidence in assessing physical and cognitive health, for example progression of neuro-degenerative diseases, quality of life, risk of fall, ability of independent living, and frailty of pre- and post-surgery patients.

### SUMMARY

The exemplary embodiments disclose a method, a structure, and a computer system for gait-based mobility analysis. The exemplary embodiments may include collecting heart rate data and acceleration data corresponding to a user while the user is not performing one or more validated fitness assessment tests and extracting one or more features from the heart rate data and the acceleration data. The exemplary in embodiments may further include calculating one or more validated fitness assessment scores of the user based on applying a model to the one or more features and projecting a mobility of the user based on the one or more validated fitness assessment scores.

### BRIEF DESCRIPTION OF THE SEVERAL VIEWS OF THE DRAWINGS

The following detailed description, given by way of example and not intended to limit the exemplary embodiments solely thereto, will best be appreciated in conjunction with the accompanying drawings, in which:

FIG. 1 depicts an exemplary schematic diagram of a mobility assessment system 100, in accordance with the exemplary embodiments.

FIG. 2A depicts an exemplary flowchart 200 illustrating the analytics pipeline of a mobility assessor 132 of the mobility assessment system 100, in accordance with the exemplary embodiments.

FIG. 2B depicts an exemplary flowchart 300 illustrating the predictive models of the mobility assessor 132 of the mobility assessment system 100, in accordance with the exemplary embodiments.

FIG. 3 depicts an exemplary block diagram depicting the hardware components of the mobility assessment system 100 of FIG. 1, in accordance with the exemplary embodiments.

FIG. 4 depicts a cloud computing environment, in accordance with the exemplary embodiments.

FIG. 5 depicts abstraction model layers, in accordance with the exemplary embodiments.

The drawings are not necessarily to scale. The drawings are merely schematic representations, not intended to portray specific parameters of the exemplary embodiments. The drawings are intended to depict only typical exemplary embodiments. In the drawings, like numbering represents like elements.

### DETAILED DESCRIPTION OF THE SEVERAL VIEWS OF THE DRAWINGS

Detailed embodiments of the claimed structures and methods are disclosed herein; however, it can be understood that the disclosed embodiments are merely illustrative of the claimed structures and methods that may be embodied in various forms. The exemplary embodiments are only illustrative and may, however, be embodied in many different forms and should not be construed as limited to the exemplary embodiments set forth herein. Rather, these exemplary embodiments are provided so that this disclosure will be thorough and complete, and will fully convey the scope to be covered by the exemplary embodiments to those skilled in the art. In the description, details of well-known features and techniques may be omitted to avoid unnecessarily obscuring the presented embodiments.

References in the specification to “one embodiment”, “an embodiment”, “an exemplary embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether or not explicitly described.

In the interest of not obscuring the presentation of the exemplary embodiments, in the following detailed description, some processing steps or operations that are known in the art may have been combined together for presentation and for illustration purposes and in some instances may have not been described in detail. In other instances, some processing steps or operations that are known in the art may not be described at all. It should be understood that the following description is focused on the distinctive features or elements according to the various exemplary embodiments.

Mobility refers to one's ability to move freely and easily. Physiologically, mobility is a manifestation of a functional integration of skeletal, muscular, nervous, circulatory, and respiratory systems. Thus, mobility may represent critical clinical evidence in assessing physical and cognitive health, for example progression of neuro-degenerative diseases, quality of life, risk of fall, ability of independent living, and frailty of pre- and post-surgery patients.

There are currently several means for analyzing user mobility that vary in complexity, scalability, and practicality. At a basic level, user mobility may be analyzed through self-reporting, for example daily or weekly questionnaires. Though inexpensive and easy to implement, self-reporting through questionnaires is lacking in both the amount/type of data collected and an accuracy thereof. Alternatively, user mobility may be assessed through clinical mobility tests such as the timed up and go (TUG), 30-second chair stand, and 6-minute walk test (6MWT). However, in addition to presenting a burden on both patients and clinicians, these methods are similarly ineffective and fail to capture all relevant data. For example, these tests fail to capture the dynamics of day-to-day mobility, which is important in understanding disease progression and therapeutic response. In addition, such tests often have a singular outcome and lack deeper insights regarding one's mobility.

Other means for analyzing user mobility include light-weight solutions, such as on-body inertial sensors that directly measure and aggregate motion of various body parts of interest. While on-body inertial sensors may accurately report motion and posture, they require complex and burdensome setups, are not suitable for monitoring longitudinal motion, lack accuracy in location and trajectory tracking, and present relatively high costs for a scalable deployment. Current mobility assessment methods may also implement infrared cameras that measure depth based on the time-of-flight (ToF) of a projected infrared laser. While the benefits of these systems include contactless sensing and the ability to reveal body details (e.g., body frame), their shortcomings include a required line of sight, and thus limited coverage/a narrow field of view, as well as subjectivity to lighting and environmental conditions.

Other mobility assessment solutions may be performed in a clinical setting where pressure mapping systems may be used to capture foot pressure of a walking user to provide a variety of gait parameters. These systems, however, are impractical for consistent use due to their complex setup and operation, as well as high cost. Similarly, more complex systems may track retro-reflective markers placed on a moving body using infrared cameras located around the clinical setting. These systems too, however, are impractical for consistent use and are not easily scaled due to their complex setup and high costs.

There is thus a need for a contactless, inexpensive, scalable, and less burdensome solution to assess various user mobility parameters consistently and accurately. Accordingly, the forthcoming detailed description presents a system for assessing user mobility via traditional methods such as the TUG and 6MWT using only a user heart rate and acceleration data gathered by, for example, a wearable device. Benefits of the proposed motion tracking system include, to name a few, low cost, scalability, and the ability to increase data collection type and rate. Moreover, benefits of the aggregation and analysis architecture further include support for a wide range of assessments, such as progression of neuro-degenerative disease, strength and endurance, cardiovascular fitness, quality of life, fall risk, frailty, etc., on a regular basis (such as daily, hourly, daily, weekly, etc.), as well as the ability to create predictive models through machine learning of the data. Detailed description of the invention follows.

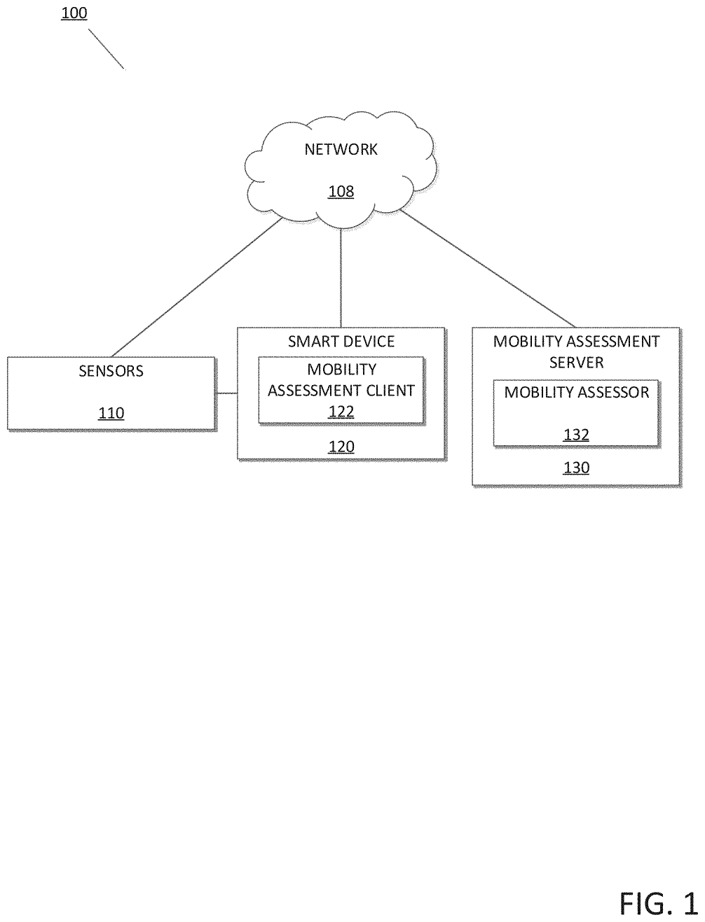

FIG. 1 depicts the gait-based mobility assessment system 100, in accordance with exemplary embodiments. According to the exemplary embodiments, the mobility assessment system 100 may include one or more sensors 110, a smart device 120, and a mobility assessment server 130, which all may be interconnected via a network 108. While programming and data of the exemplary embodiments may be stored and accessed remotely across several servers via the network 108, programming and data of the exemplary embodiments may alternatively or additionally be stored locally on as few as one physical computing device or amongst other computing devices than those depicted. The operations of the mobility assessment system 100 are described in greater detail herein.

In the exemplary embodiments, the network 108 may be a communication channel capable of transferring data between connected devices. In the exemplary embodiments, the network 108 may be the Internet, representing a worldwide collection of networks and gateways to support communications between devices connected to the Internet. Moreover, the network 108 may utilize various types of connections such as wired, wireless, fiber optic, etc., which may be implemented as an intranet network, a local area network (LAN), a wide area network (WAN), a combination thereof, etc. In further embodiments, the network 108 may be a Bluetooth network, a Wi-Fi network, a combination thereof, etc. The network 108 may operate in frequencies including 2.4 gHz and 5 gHz internet, near-field communication, etc. In yet further embodiments, the network 108 may be a telecommunications network used to facilitate telephone calls between two or more parties comprising a landline network, a wireless network, a closed network, a satellite network, a combination thereof, etc. In general, the network 108 may represent any combination of connections and protocols that will support communications between connected devices.

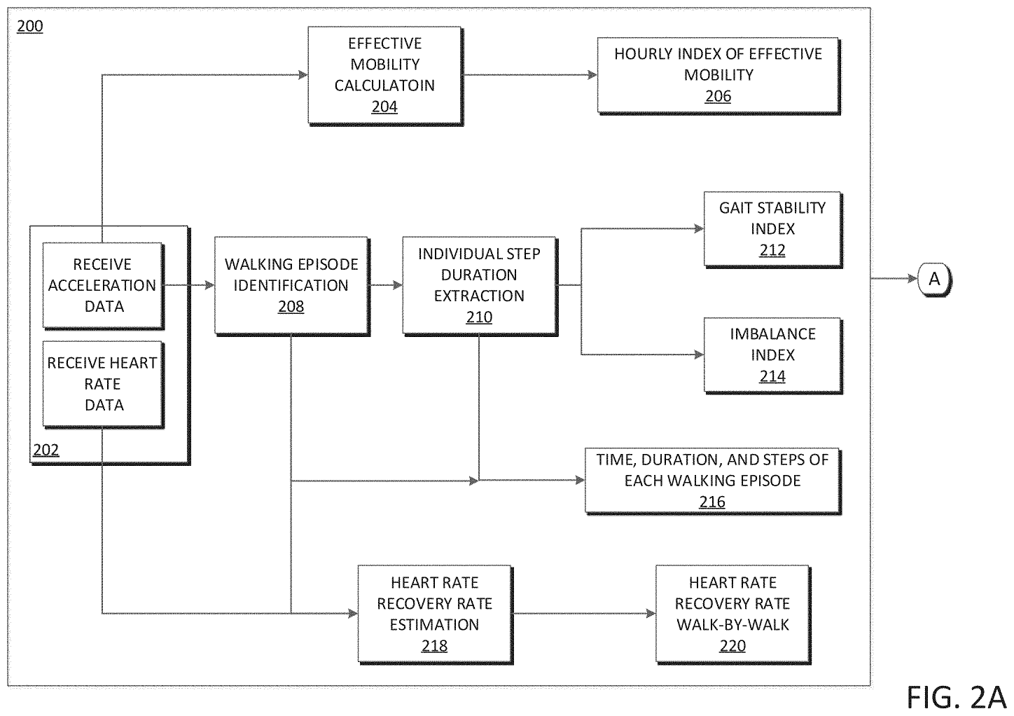

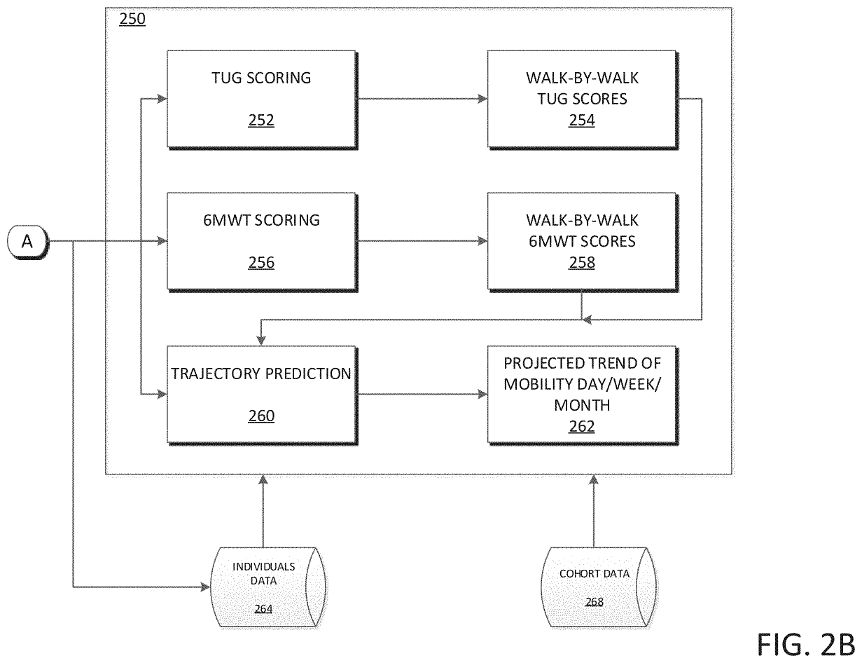

In exemplary embodiments, the sensors 110 may be one or more devices, e.g., wearable devices, capable of collecting data. In particular, the sensors 110 may be configured to collect data that may be analysed to estimate a motion and mobility of a user, including acceleration, heartrate, location, center of mass, body frame, user state, body orientation, skeleton, joints, ECG (electrocardiogram) signal, EMG (electromyography) signal, PPG (Photoplethysmography) signal, Blood oxygen saturation, etc. Accordingly, the sensors 110 may be a wearable smart device (e.g., a watch), adhesive patch, smart clothes, etc., that includes an accelerometer, heartrate monitor, gyroscope, electrodes (e.g., electrocardiogram/electromyography/electrodes), LED (e.g., photoplethysmography LEDS), a Global Positioning System (GPS), etc. In embodiments, the sensors 110 may communicate with the network 108 or with the smart device 120 through means such as WiFi, Bluetooth, Near Field Communication (NFC), etc. In general, the sensors 110 may be any device capable of collecting data relating to acceleration and heart rate of a wearer. The sensors 110 are described in greater detail with respect to FIG. 2-5.

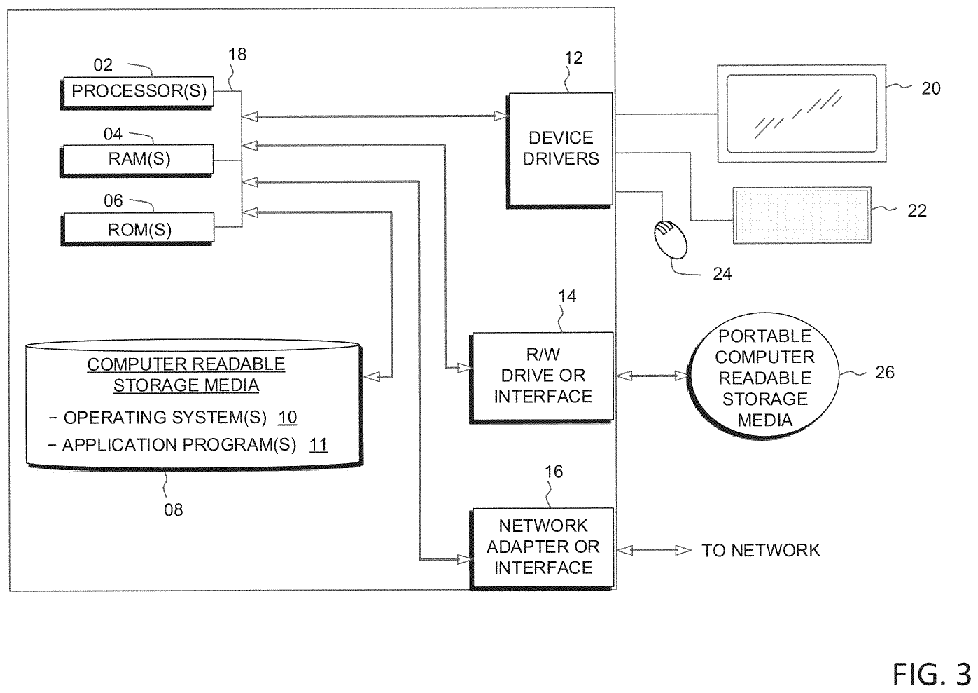

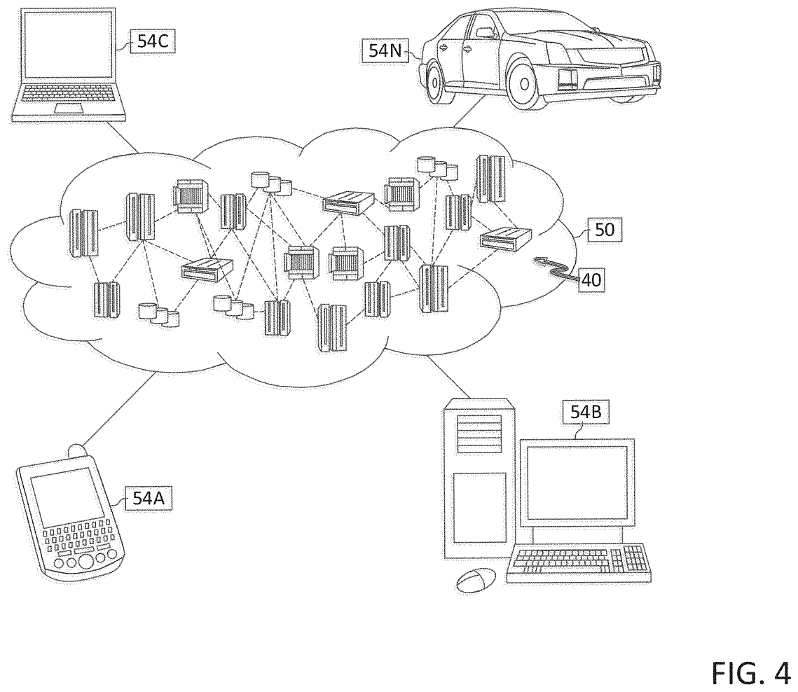

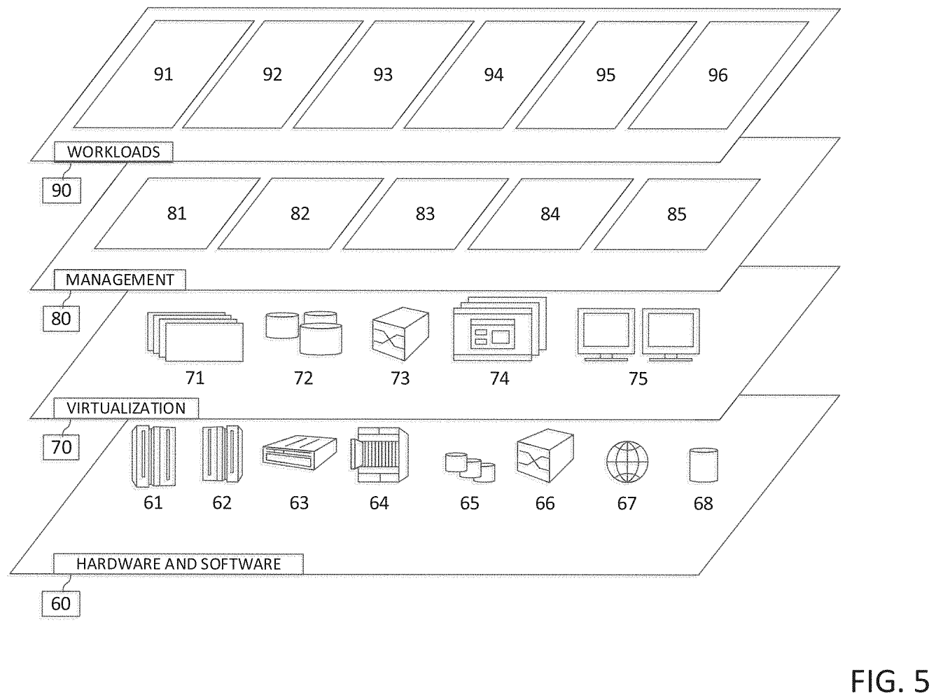

In exemplary embodiments, the smart device 120 includes a mobility assessment client 122, and may be an enterprise server, a laptop computer, a notebook, a tablet computer, a netbook computer, a personal computer (PC), a desktop computer, a server, a personal digital assistant (PDA), a smart phone, a mobile phone, a virtual device, a thin client, an IoT device, or any other electronic device or computing system capable of sending and receiving data to and from other computing devices. While the smart device 120 is shown as a single device, in other embodiments, the smart device 120 may be comprised of a cluster or plurality of computing devices, in a modular manner, etc., working together or working independently. The smart device 120 is described in greater detail as a hardware implementation with reference to FIG. 3, as part of a cloud implementation with reference to FIG. 4, and/or as utilizing functional abstraction layers for processing with reference to FIG. 5.

The mobility assessment client 122 may act as a client in a client-server relationship, and may be a software and/or hardware application capable of communicating with and providing a user interface for a user to interact with the mobility assessment server and other computing devices via the network 108. Moreover, the mobility assessment client 122 may be further capable of transferring data from the smart device 120 to and from other devices via the network 108. In embodiments, the mobility assessment client 122 may utilize various wired and wireless connection protocols for data transmission and exchange, including Bluetooth, 2.4 gHz and 5 gHz internet, near-field communication (NFC), etc. The mobility assessment client 122 is described in greater detail with respect to FIG. 2-5.

In exemplary embodiments, the mobility assessment server 130 includes a mobility assessor 132, and may act as a server in a client-server relationship with the mobility assessment client 122. The mobility assessment server 130 may be an enterprise server, a laptop computer, a notebook, a tablet computer, a netbook computer, a personal computer (PC), a desktop computer, a server, a personal digital assistant (PDA), a smart phone, a mobile phone, a virtual device, a thin client, an IoT device, or any other electronic device or computing system capable of sending and receiving data to and from other computing devices. While the mobility assessment server 130 is shown as a single device, in other embodiments, the mobility assessment server 130 may be comprised of a cluster or plurality of computing devices, in a modular manner, etc., working together or working independently. The mobility assessment server 130 is described in greater detail as a hardware implementation with reference to FIG. 3, as part of a cloud implementation with reference to FIG. 4, and/or as utilizing functional abstraction layers for processing with reference to FIG. 5.

The mobility assessor 132 may be a software and/or hardware program that may perform an analytics pipeline (FIG. 2A, 200) and generate one or more predictive models (FIG. 2B, 250).

In particular, and with respect to performing the analytics pipeline (FIG. 2A, 200), the mobility assessor 132 may receive acceleration and heart rate data. Based on the acceleration data, the mobility assessor 132 may further perform an effective mobility calculation, as well as determine an hourly index of effective mobility. Similarly based on the acceleration data, the mobility assessor 132 may identify walking episodes and extract individual step durations of those walking episodes. Based thereon, the mobility assessor 132 may determine a gait stability index as well as an imbalance index. The mobility assessor 132 may then determine a time, duration, and number of steps of each walking episode based on the identified walking episodes and the individual step duration. Lastly, the mobility assessor 132 may determine a heart rate recovery estimation and heart rate recovery wake walk-by-walk.

Based on performing the analytics pipeline described above, and turning now to the mobility assessor 132 generating one or more predictive models (FIG. 2B, 250), the mobility assessor 132 may perform a TUG scoring and determine walk-by-walk TUG scores. In addition, the mobility assessor 132 may perform a 6MWT scoring and determine walk-by-walk 6MWT scores. The mobility assessor 132 may lastly perform a trajectory prediction and determine a projected trend of mobility. The mobility assessor 132 is described in greater detail with reference to FIG. 2-5.

FIG. 2A depicts an exemplary flowchart 200 illustrating the analytics pipeline of a mobility assessor 132 of the mobility assessment system 100, in accordance with the exemplary embodiments. In exemplary embodiments, the mobility assessor 132 may be initially configured by first receiving user consent to collect data as well as registration information based on, for example, log in credentials, internet protocol (IP) address, media access control (MAC) address, etc., via the mobility assessment client 122 and the network 108. With respect to receiving user consent, the mobility assessment client 122 may allow a user to manage the data collected and the manner in which the data may be collected, used, transferred, distributed, etc., as well as an option to opt out of such data collection. In any managing of user data, the mobility assessor 132 may be configured to adhere to at least all data handling and privacy protocols applicable.

In addition to receiving user consent, the mobility assessor 132 may further receive user registration information, including demographic information, such as user name, date of birth, location, etc., as well as health and mobility related data. The health and mobility related data may be received via user/physician input, reference to an electronic health/medical record, etc., and may include one or more user health conditions, baseline user metrics, etc. Configuration may also include establishing communication with the sensors 110 via, e.g., WiFi, Bluetooth, or NFC.

The mobility assessor 132 may receive user acceleration and heart rate data (step 202). In embodiments, the mobility assessor 134 may receive acceleration and heart rate data via communication with the sensors 110 via the network 108. In embodiments, and depending on the wearable device in which the sensors 110 are integrated (e.g., a smart watch), the data may be received first by the smart device 120 (e.g., via NFC) before transmission to the mobility assessor 132. The acceleration data, herein defined as rate of change in velocity over time, may be in any suitable rate format, and may be received in one or more axis, e.g., x, y, and z coordinate planes. Similarly, the heart rate data may be in any suitable rate format, e.g., beats per minute (BPM).

In order to better illustrate the operations of the mobility assessor 132, reference is now made to an illustrative example wherein a user consents to having both heart rate and acceleration data collected prior to linking a smart watch to collect such data.

The mobility assessor 132 may calculate an effective mobility (step 204). In embodiments, an effective mobility captures an over amplitude of user motion, and may be determined by performing a spectrum analysis of the received acceleration data. More specifically, the mobility assessor 132 may first calculate the activity intensity (AI) as:

$$AI = \sqrt{\frac{( {σ ( a\_x )}^{2} + {σ ( a\_y )}^{2} + {σ ( a\_z )}^{2} )}{3}} * 100 \qquad Eq . 1$$

Where a_x is acceleration in the x axis, a_y is acceleration in the y axis, and a_z is acceleration in the z axis. In embodiments, the mobility assessor 132 may use data from, e.g., a 30 seconds window, every 15 seconds. The mobility assessor 132 may then determine time spent in minutes within the following states, which are based on certain thresholds observed during mobility assessment tests for the user or across several patients over multiple visits:

Rest if AI<=10

Low if AI>10 & AI<=50

Low-Med if AI>50 & AI<=150

Med if AI>150 & AI<=250

Med-High if AI>250 & AI<=400

High if AI>400

The effective mobility (EM) may then be defined by:

$$EM = \frac{\frac{\frac{\begin{matrix}( rest * 0 + low * 1 + low - med * 2 + \\ med * 3 + med - high * 4 + high * 5 )\end{matrix}}{15}}{60}}{24} \qquad Eq . 2$$

Where rest is minutes spent per day at rest AI, low is minutes spent per day at low AI, low-med is minutes spent per day at low-med AI, med is minutes spent per day at med AI, med-high is minutes spent per day at med-high AI, and high is minutes spent per day at high AI.

Furthering the illustrative example introduced above, the mobility assessor 132 may determine an effective mobility of the user based on determining minutes spent per day in different activity intensities from the accelerometer data collected by the user's smart watch.

The mobility assessor 132 may calculate an hourly index of effective mobility (step 206). In embodiments, the hourly index of effective mobility indicates an objective level of user mobility per hour, and may be determined based on breaking down the effective mobility calculation into a per hour determination. The mobility assessor 132 may, e.g., use a time series of the acceleration data in order to isolate different hours of the day and discern an hourly index of effective mobility. The hourly index of mobility may indicate variability in times at which a user is mobile.

With reference again to the formerly introduced example, the mobility assessor 132 may calculate an hourly index of effective mobility for the user based on isolating the effective mobility data on a per hour basis.

The mobility assessor 132 may identify one or more walking episodes (step 208). In exemplary embodiments, the mobility assessor 132 may identify walking episodes based on the received acceleration data. In particular, the mobility assessor 132 may identify walking episodes by identifying specific patterns in the acceleration data, e.g., rapid increases or decreases in acceleration, a cadence in user acceleration, GPS locational data, etc. In embodiments, walking episode detection involves the following process. First, identify periods of significant motion in the accelerometer signal by, e.g., computing the variance in the accelerometer signal within a rolling processing time window and comparing whether the computed variance is above a certain threshold. For example, the threshold could be 0.05 g where g=9.8 m/s2. Next, if the mobility assessor 132 detects significant motion, zero-mean the signal by subtracting the mean of the signal from the signal. Next, the mobility assessor 132 may check for periodic peaks and troughs in zero-mean signal. If the mobility assessor 132 detects peaks and troughs at a rate of ˜0.2-2 Hz, for a duration of ˜30 seconds, then the identified period of significant motion is characterized as a walking episode. In embodiments, the mobility assessor 132 may additionally identify a change in a location of the user via GPS coordinates at a particular rate in order to infer that the user is ambulating. Similarly, the mobility assessor 132 may identify an increase in heart rate indicative of ambulation. Overall, the mobility assessor 132 may utilize any means for identifying a walking episode of the user.

Continuing the earlier introduced example, the mobility assessor 132 identifies a walking episode of the user based on detecting patterns in acceleration data indicative of user ambulation.

The mobility assessor 132 may extract individual step durations (step 210). The individual step duration refers to the time duration, e.g., in seconds, when right leg is used to walk as opposed to when left leg is used to walk. In embodiments, the difference between the right and left step duration may be used as a feature indicative of gait stability of the user. The mobility assessor 132 may determine step durations based on the acceleration data and identified walking episodes, with specific patterns in acceleration and heart rate indicative of walking with each leg. More specifically, the mobility assessor 132 may identify points at which using one leg, e.g., right or left, starts making contact with the ground and ceases making contact with the ground based on identifying moments of increased and decreased acceleration. For example, the mobility assessor 132 may identify a rapid decrease/stop in acceleration when a foot hits the ground. Conversely, the mobility assessor 132 may detect a rapid change in acceleration as a foot reaches the apex of the walking motion and begins to then travel downward. The mobility assessor 132 may further identify acceleration in lateral motions, e.g., horizontal acceleration to the right when stepping onto the right leg. Overall, the mobility assessor 132 may utilize the accelerometer and heartrate data in order to identify individual step durations via any suitable means.

With reference to the previously introduced example, the mobility assessor 132 identifies individual step durations of each foot (in seconds) during a walking episode based on the received accelerometer data.

The mobility assessor 132 may calculate a gait stability index (step 212). In exemplary embodiments, the gait stability index is a measure of how the center of gravity of a user moves horizontally while walking and may be extracted by analysing the durations of each of the individual left and right steps during a walking episode. In particular, the mobility assessor 132 may calculate a stability index based on determining a difference in the individual step duration, where a shorter difference between the right and left step durations is indicative of a more stable user. Conversely, a longer difference between the right and left step duration indicates a less stable user. Based on the difference in step duration, the mobility assessor 132 may then compute the gait stability index by identifying a gait stability index value that corresponds to the difference in step duration. Corresponding gait stability index values may, e.g., be based on data corresponding to the user, other users, other users within a similar cohort, etc.

In furthering the example introduced above, the mobility assessor 132 determines a gait stability index value based on the difference between the step duration of the right and left legs of the user

The mobility assessor 132 may calculate an imbalance index (step 214). In exemplary embodiments, the imbalance index is a measure of how the center of gravity of a user moves vertically while a user is walking, and may be extracted from the identified walking episodes and durations of each of the individual left and right steps during a walking episode. In particular, the mobility assessor 132 may determine an imbalance index by measuring an amount of vertical movement/acceleration during each step.

In furthering the example introduced above, the mobility assessor 132 determines an imbalance index based on determining vertical movement on the user during a walking episode.

The mobility assessor 132 may determine a time, duration, and steps of each walking episode (step 216). In embodiments, the mobility assessor 132 may determine the time, duration, and steps of each walking episodes in order to identify candidates that can be used to simulate gait-based mobility tests and estimate their scores. In particular, the mobility assessor 132 may utilize walking episodes as data from which to estimate mobility in clinically accepted and validated mobility tests, for example the TUG and 6MWT. Accordingly, the mobility assessor 132 may identify such walking episodes for input into a model correlating walking episodes with validated mobility tests, as will be described in greater detail forthcoming.

Returning to the example above, the mobility assessor 132 identifies a time, duration, and steps of each walking episode of the user.

The mobility assessor 132 may estimate a heart rate recover rate (step 218). At the conclusion of walking episodes, the heart rate will recover to the baseline heart rate, which can be recorded in order to capture functional performance following a walking episode. The mobility assessor 132 may assess heart rate recover rate by first estimating a dynamic baseline and peak heart rate based on the received heart rate data and identified walking episodes. In particular, the mobility assessor 132 may estimate dynamic baseline heart rates based on received heart rates at times of low user activity/rest as determined by the accelerometer data, e.g., at times outside of walking episodes. Conversely, the mobility assessor 132 may identify a peak heart rate as a maximum heart rate recorded, most likely during high activity. The estimated baseline heart rate, peak heart rate, and heart rate recovery may be dynamic in that the mobility assessor 132 may adaptively change the estimation over time.

In furthering the example introduced above, the mobility assessor 132 determines an estimated heart rate recovery by comparing the peak heart rate during high activity to the baseline heart rate of the user.

The mobility assessor 132 may calculate a heart rate recovery rate walk-by-walk (step 220). As used herein, walk-by-walk merely implies metrics per walking episode, and the mobility assessor 132 may determine a heart rate recovery walk-by-walk based on the heart rate recovery rate over the identified one or more walking episodes. In embodiments, the mobility assessor 132 considers a faster heart rate recovery rate (e.g., ˜50 beats per minute (BPM) as an indication of good health and an ideal fitness assessment score, while a slow heart rate recovery rate (e.g., ˜10 BPM) is considered an indication of poor health and poor fitness assessment scores. The mobility assessor 132 may be configured to determine fitness scores based on heart rate recovery rate at any granularity, e.g., a ranking system of ideal/poor, 1-10, etc.

With reference to the previously introduced example, the mobility assessor 132 determines a heart rate recovery rate walk-by-walk of the user based on the estimated heart rate recovery rate and identified walking episodes.

FIG. 2B depicts an exemplary flowchart 300 illustrating the predictive models of the mobility assessor 132 of the mobility assessment system 100, in accordance with the exemplary embodiments.

The mobility assessor 132 may perform a TUG scoring (step 252). In exemplary embodiments, the mobility assessor 132 may perform a TUG scoring based on training a model that correlates TUG scoring with features extracted from the acceleration and heart rate data above, e.g., the hourly index of effective mobility, stability index, imbalance index, time, duration, and steps of each walking episode, and heart rate recovery rate walk-by-walk. In particular, the mobility assessor 132 may first train a model by prompting a user to perform validated fitness assessment motions, e.g., a TUG, while acceleration and heart rate data is collected and the features identified above are extracted. The validated fitness assessment motion may then be scored and the mobility assessor 132 may train the model to correlate the extracted features with the validated fitness assessment scores. Once trained, the model may then compare features extracted in real time with those correlated to the validated fitness assessment scores and deduce a real-time validated fitness score therefrom. For example, a shorter ‘step duration’ value would predict a TUG score to be smaller˜8 sec, while a longer ‘step duration’ value would predict the TUG score to be larger˜10-12 sec. Similarly, a shorter ‘step duration’ value would predict the 6MWT score to be higher and vice versa. Overall, a current validated fitness assessment score may be deduced based on a comparison of real time feature values to those known during a scored validated fitness assessment test. Following the training phase of the model in which a user performs a TUG while features are extracted, the mobility assessor 132 may no longer require a user to perform a TUG, but rather TUG scores may be inferred from the acceleration and heart rate features. In particular, the mobility assessor 132 may extract real-time features for comparison to features having known TUG scores and, based on comparing the features, deduce a new TUG score.

With reference to the previously introduced example, the mobility assessor 132 computes a TUG scores based on comparing the heart rate- and acceleration-based feature values of known TUG scores to those feature values currently exhibited in order to deduce a current TUG score.

The mobility assessor 132 may calculate walk-by-walk TUG scores (step 254). In exemplary embodiments, the mobility assessor 132 may calculate walk-by-walk TUG scores by calculating TUG scores for each walking episode.

In furthering the example introduced above, the mobility assessor 132 computes walk-by-walk TUG scoring based on the TUG scoring and the times at which the TUG scoring data is collected.

The mobility assessor 132 may perform a 6MWT scoring (step 256). In embodiments, the mobility assessor 132 may perform a 6MWT scoring similar to performing the TUG scoring above in that a user is first prompted to perform a 6MWT while acceleration and heart rate data is gathered and features are extracted, namely the hourly index of effective mobility, stability index, imbalance index, time, duration, and steps of each walking episode, and heart rate recovery rate walk-by-walk. The mobility assessor 132 may then deduce current 6MWT scores based on comparison of current feature values to those exhibited during the 6MWT having known 6MWT scores.

With reference to the previously introduced example, the mobility assessor 132 computes a 6MWT scoring based on the heart rate- and acceleration-based features.

The mobility assessor 132 may calculate walk-by-walk 6MWT scores (step 258). In exemplary embodiments, the mobility assessor 132 the mobility assessor 132 may calculate walk-by-walk 6MWT scores by calculating 6MWT scores for each walking episode and the times at which the 6MWT scoring data is collected.

In furthering the example introduced above, the mobility assessor 132 computes a walk-by-walk 6MWT scoring based on the 6MWT scoring.

It should be noted that in addition to the TUG and 6MWT, the present invention is equally applicable to other validated and/or clinically approved fitness assessment tests. For example, using a similar process to that of training the model for the TUG and 6MWT above, the mobility assessor 132 may be equally capable of similarly inferring scores for a Two Minute Walk Test (2MWT), 3 Minute Walk Test (3MWT), Ten Minute Walk Test (10MWT), Tandem Walking (TW), Two Minute Step in Place and Test, Sit-to-Stand.

It will be further appreciate by one skilled in the art that individual and cohort data may be further included in the aforementioned modelling. Individual and cohort data may include demographics, body mass index (BMI), race, ethnicity, etc., and cohorts may be defined based on age, gender, ethnicity, comorbidity, disease condition, etc. The present invention may further consider individual and cohort data in determining fitness assessment test scores. For example, TUG scores can increase with age and 6MWT scores can decrease with age in certain cohort of patients. Accordingly, modelling user fitness assessment scores may further include such tendencies of the cohort or user in particular.

The mobility assessor 132 may perform a trajectory prediction (step 260). In embodiments, the mobility assessor 132 may perform a trajectory prediction based on whether the scores of the user are improving or worsening. In embodiments, the mobility assessor 132 may base the trajectory prediction based exclusively on the TUG and/or 6MWT scores while, in other embodiments, the mobility assessor 132 may additionally or alternatively consider multiple fitness assessment tests.

In furthering the previously introduced example, the mobility assessor 132 compares a TUG and 6MWT scoring of the user to historic TUG and 6MWT scores of the user.

The mobility assessor 132 may estimate projected trends of mobility (step 262). In embodiments, the mobility assessor 132 may predict trends of mobility based on the trajectory prediction, namely whether the scoring of the user is improving or worsening.

Concluding the aforementioned example, the mobility assessor 132 may project an increase in mobility based on the scoring associated with the user improving over time.

FIG. 3 depicts a block diagram of devices used within mobility assessment system 100 of FIG. 1, in accordance with the exemplary embodiments. It should be appreciated that FIG. 5 provides only an illustration of one implementation and does not imply any limitations with regard to the environments in which different embodiments may be implemented. Many modifications to the depicted environment may be made.

Devices used herein may include one or more processors 02, one or more computer-readable RAMs 04, one or more computer-readable ROMs 06, one or more computer readable storage media 08, device drivers 12, read/write drive or interface 14, network adapter or interface 16, all interconnected over a communications fabric 18. Communications fabric 18 may be implemented with any architecture designed for passing data and/or control information between processors (such as microprocessors, communications and network processors, etc.), system memory, peripheral devices, and any other hardware components within a system.

One or more operating systems 10, and one or more application programs 11 are stored on one or more of the computer readable storage media 08 for execution by one or more of the processors 02 via one or more of the respective RAMs 04 (which typically include cache memory). In the illustrated embodiment, each of the computer readable storage media 08 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Devices used herein may also include a RAY drive or interface 14 to read from and write to one or more portable computer readable storage media 26. Application programs 11 on said devices may be stored on one or more of the portable computer readable storage media 26, read via the respective RAY drive or interface 14 and loaded into the respective computer readable storage media 08.

Devices used herein may also include a network adapter or interface 16, such as a TCP/IP adapter card or wireless communication adapter (such as a 4G wireless communication adapter using OFDMA technology). Application programs 11 on said computing devices may be downloaded to the computing device from an external computer or external storage device via a network (for example, the Internet, a local area network or other wide area network or wireless network) and network adapter or interface 16. From the network adapter or interface 16, the programs may be loaded onto computer readable storage media 08. The network may comprise copper wires, optical fibers, wireless transmission, routers, firewalls, switches, gateway computers and/or edge servers.

Devices used herein may also include a display screen 20, a keyboard or keypad 22, and a computer mouse or touchpad 24. Device drivers 12 interface to display screen 20 for imaging, to keyboard or keypad 22, to computer mouse or touchpad 24, and/or to display screen 20 for pressure sensing of alphanumeric character entry and user selections. The device drivers 12, R/W drive or interface 14 and network adapter or interface 16 may comprise hardware and software (stored on computer readable storage media 08 and/or ROM 06).

The programs described herein are identified based upon the application for which they are implemented in a specific one of the exemplary embodiments. However, it should be appreciated that any particular program nomenclature herein is used merely for convenience, and thus the exemplary embodiments should not be limited to use solely in any specific application identified and/or implied by such nomenclature.

Based on the foregoing, a computer system, method, and computer program product have been disclosed. However, numerous modifications and substitutions can be made without deviating from the scope of the exemplary embodiments. Therefore, the exemplary embodiments have been disclosed by way of example and not limitation.

It is to be understood that although this disclosure includes a detailed description on cloud computing, implementation of the teachings recited herein are not limited to a cloud computing environment. Rather, the exemplary embodiments are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or data center).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 50 is depicted. As shown, cloud computing environment 50 includes one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 40 and cloud computing environment 50 can communicate with any type of computerized device over any type of network and/or network addressable connection (e.g., using a web browser).

Referring now to FIG. 5, a set of functional abstraction layers provided by cloud computing environment 50 (FIG. 4) is shown. It should be understood in advance that the components, layers, and functions shown in FIG. 5 are intended to be illustrative only and the exemplary embodiments are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and mobility processing 96.

The exemplary embodiments may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. A computer-implemented method for assessing user mobility, the method comprising:
collecting heart rate data and acceleration data corresponding to a user while the user is not performing one or more validated fitness assessment tests;
extracting one or more features from the heart rate data and the acceleration data;
calculating one or more validated fitness assessment scores of the user based on applying a model to the one or more features; and
projecting a mobility of the user based on the one or more validated fitness assessment scores.

2. The method of claim 1, wherein the one or more validated fitness assessment tests are selected from a group consisting of a Timed-Up and Go (TUG), Six Minute Walk Test (6MWT), Two Minute Walk Test (2MWT), 3 Minute Walk Test (3MWT), Ten Minute Walk Test (10MWT), Tandem Walking (TW), Two Minute Step in Place Test, and Sit-to-Stand.

3. The method of claim 1, wherein the one or more features are selected from a group comprising an hourly index of effective mobility, a gait stability index, an imbalance index, a heart rate recovery rate walk-by-walk, and a time, duration, and steps of one or more walking episodes.

4. The method of claim 3, wherein the gait stability index, the imbalance index, and the time, duration, and steps of the one or more walking episodes are calculated by extracting one or more individual step durations from the one or more walking episodes.

5. The method of claim 3, wherein the heart rate recovery rate walk-by-walk is calculated by estimating a baseline and a peak heart rate of the user based on the heart rate data.

6. The method of claim 1, wherein the model is trained by correlating the one or more features with the one or more validated fitness assessment scores while the user performs the one or more corresponding validated fitness assessment tests.

7. The method of claim 1, wherein the projected mobility of the user is based on comparing the one or more validated fitness assessment scores to one or more historic validated fitness assessment scores.

8. A computer program product for assessing user mobility, the computer program product comprising:
one or more non-transitory computer-readable storage media and program instructions stored on the one or more non-transitory computer-readable storage media capable of performing a method, the method comprising:
collecting heart rate data and acceleration data corresponding to a user while the user is not performing one or more validated fitness assessment tests;
extracting one or more features from the heart rate data and the acceleration data;
calculating one or more validated fitness assessment scores of the user based on applying a model to the one or more features; and
projecting a mobility of the user based on the one or more validated fitness assessment scores.

9. The computer program product of claim 8, wherein the one or more validated fitness assessment tests are selected from a group consisting of a Timed-Up and Go (TUG), Six Minute Walk Test (6MWT), Two Minute Walk Test (2MWT), 3 Minute Walk Test (3MWT), Ten Minute Walk Test (10MWT), Tandem Walking (TW), Two Minute Step in Place Test, and Sit-to-Stand.

10. The computer program product of claim 8, wherein the one or more features are selected from a group comprising an hourly index of effective mobility, a gait stability index, an imbalance index, a heart rate recovery rate walk-by-walk, and a time, duration, and steps of one or more walking episodes.

11. The computer program product of claim 10, wherein the gait stability index, the imbalance index, and the time, duration, and steps of the one or more walking episodes are calculated by extracting one or more individual step durations from the one or more walking episodes.

12. The computer program product of claim 10, wherein the heart rate recovery rate walk-by-walk is calculated by estimating a baseline and a peak heart rate of the user based on the heart rate data.

13. The computer program product of claim 8, wherein the model is trained by correlating the one or more features with the one or more validated fitness assessment scores while the user performs the one or more corresponding validated fitness assessment tests.

14. The computer program product of claim 8, wherein the projected mobility of the user is based on comparing the one or more validated fitness assessment scores to one or more historic validated fitness assessment scores.

15. A computer system for assessing user mobility, the system comprising:
one or more computer processors, one or more computer-readable storage media, and program instructions stored on the one or more of the computer-readable storage media for execution by at least one of the one or more processors capable of performing a method, the method comprising:
collecting heart rate data and acceleration data corresponding to a user while the user is not performing one or more validated fitness assessment tests;
extracting one or more features from the heart rate data and the acceleration data;
calculating one or more validated fitness assessment scores of the user based on applying a model to the one or more features; and
projecting a mobility of the user based on the one or more validated fitness assessment scores.

16. The computer system of claim 15, wherein the one or more validated fitness assessment tests are selected from a group consisting of a Timed-Up and Go (TUG), Six Minute Walk Test (6MWT), Two Minute Walk Test (2MWT), 3 Minute Walk Test (3MWT), Ten Minute Walk Test (10MWT), Tandem Walking (TW), Two Minute Step in Place Test, and Sit-to-Stand.

17. The computer system of claim 15, wherein the one or more features are selected from a group comprising an hourly index of effective mobility, a gait stability index, an imbalance index, a heart rate recovery rate walk-by-walk, and a time, duration, and steps of one or more walking episodes.

18. The computer system of claim 17, wherein the gait stability index, the imbalance index, and the time, duration, and steps of the one or more walking episodes are calculated by extracting one or more individual step durations from the one or more walking episodes.

19. The computer system of claim 17, wherein the heart rate recovery rate walk-by-walk is calculated by estimating a baseline and a peak heart rate of the user based on the heart rate data.

20. The computer system of claim 15, wherein the model is trained by correlating the one or more features with the one or more validated fitness assessment scores while the user performs the one or more corresponding validated fitness assessment tests.
